(19) United States
(12) Patent Application Publication
(10) Pub. No.: US 2010/0160370 A1
(43) Pub. Date: Jun. 24, 2010

(54) METHOD OF TREATMENT AND PHARMACEUTICAL COMPOSITIONS

(75) Inventors: Howard S. Jaffe, Mill Valley, CA (US); Hans C. Reiser, San Francisco, CA (US); Clifford Dean Wright, Sammamish, WA (US)

Correspondence Address: GILEAD SCIENCES INC, 333 LAKESIDE DR, FOSTER CITY, CA, 94404, US

(73) Assignee: Gilead Sciences, Inc., Foster City, CA (US)

(21) Appl. No.: 12/487,890

(22) Filed: Jun. 19, 2009

Related U.S. Application Data
(60) Provisional application No. 61/080,181, filed on Jul. 11, 2008.
(60) Provisional application No. 61/083,372, filed on Jul. 24, 2008.
(60) Provisional application No. 61/138,204, filed on Dec. 17, 2008.

(51) Int. Cl. A61K 31/4725; A61P 11/00

(52) U.S. Cl. 514/307

(57) ABSTRACT

The present invention relates to methods, uses, and compositions comprising the caspase inhibitor (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof.

### RELATED APPLICATIONS

This claims priority to U.S. provisional application Nos. 61/080,181, filed Jul. 11, 2008; 61/083,372, filed Jul. 24, 2008; and 61/138,204, filed Dec. 17, 2008.

### FIELD OF THE INVENTION

The present invention relates to methods, uses, and compositions comprising the caspase inhibitor (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, or a pharmaceutically acceptable salt thereof.

### BACKGROUND OF THE INVENTION

Scarring is the body's normal wound healing response in which specialized cells called fibroblasts deposit layers of collagen, a ubiquitous protein that helps form a scar. Sometimes the normal wound healing response goes awry, and the formation of scar tissue occurs faster than collagen is naturally broken down. The excessive production and deposition of collagen results in pathological scarring, a process called fibrosis.

Fibrosis is the formation or development of excess fibrous connective tissue in an organ or tissue as a reparative or reactive process, as opposed to a formation of fibrous tissue as a normal constituent of an organ or tissue. Fibrosis, much like inflammation, is one of the major, classic pathological processes in medicine. Recognized types of fibrosis include cystic fibrosis of the pancreas and lungs, injection fibrosis, which can occur as a complication of intramuscular injections, especially in children, endomyocardial fibrosis, idiopathic pulmonary fibrosis of the lung, mediastinal fibrosis, myelofibrosis, retroperitoneal fibrosis, progressive massive fibrosis, a complication of coal workers' pneumoconiosis, and nephrogenic systemic fibrosis. Conditions often associated with cirrhosis can result from fibrosis of the liver, diffuse parenchymal lung disease, post-vasectomy pain syndrome, tuberculosis (TB) can cause fibrosis of the lungs, sickle-cell anemia may cause enlargement and ultimately fibrosis of the spleen, and rheumatoid arthritis. Thus, fibrosis is a key component of multiple diseases that affect millions of people worldwide including: idiopathic pulmonary fibrosis (lung fibrosis of unknown origin); scleroderma (thickening of the skin); diabetic retinopathy and age-related macular degeneration (fibrotic diseases of the eye and leading causes of blindness); diabetic nephropathy, glomerulosclerosis and IgA nephropathy (causes of kidney failure and the need for dialysis and retransplant); cirrhosis and biliary atresia (leading causes of liver fibrosis and failure), and congestive heart failure.

Pulmonary fibrosis involves scarring of the lung. Gradually, the air sacs of the lungs become replaced by fibrotic tissue. When the scar forms, the tissue becomes thicker causing an irreversible loss of the tissue's ability to transfer oxygen into the bloodstream. Interstitial lung disease (ILD) is a general term that includes a variety of chronic lung disorders. When a person has ILD, the lung is affected in three ways. First, the lung tissue is damaged in some known or unknown way. Second, the walls of the air sacs in the lung become inflamed. Finally, fibrosis (scarring) begins in the interstitium (tissue between the air sacs), causing the lungs to become stiff. Some types of ILD have known causes while others (idiopathic) do not have a known cause.

Interstitial lung disease (ILD) is a broad category of lung diseases that includes more than 130 disorders characterized by scarring (fibrosis) and/or inflammation of the lungs. ILD accounts for 15% of the cases seen by pulmonologists (lung specialists). Some of the disorders included under the heading of ILD are: Idiopathic pulmonary fibrosis (IPF); Connective tissue or autoimmune disease-related pulmonary fibrosis; Hypersensitivity pneumonitis; Sarcoidosis; Eosinophilic granuloma, also known as Langerhan's cell histiocytosis, Chronic eosinophilic pneumonia. Wegener's granulomatosis, Idiopathic pulmonary hemosiderosis; Bronchiolitis obliterans; and Lymphangioleiomyomatosis

Idiopathic Pulmonary Fibrosis (IPF), also known as cryptogenic fibrosing alveolitis, is a chronic, progressive interstitial lung disease with an unknown cause. IPF is defined as a distinctive type of chronic fibrosing interstitial pneumonia of unknown cause associated with a histological pattern of usual interstitial pneumonia (UIP). It is perhaps better characterized, however, as an abnormal and excessive deposition of fibrotic tissue in the pulmonary interstitium with minimal associated inflammation.

Idiopathic pulmonary fibrosis is a type of idiopathic interstitial pneumonia (IIP), which as noted above, in turn is a type (or group) of interstitial lung diseases. Idiopathic interstitial pneumonias include idiopathic pulmonary fibrosis (IPF), nonspecific interstitial pneumonia, cryptogenic organizing pneumonia, acute interstitial pneumonia, respiratory bronchiolitis-associated interstitial lung disease, desquamative interstitial pneumonia, and lymphoid interstitial pneumonia.

The prevalence of IPF has been estimated to be over 50,000 cases in the U.S., with an annual incidence of approximately 15,000. There are no FDA-approved treatments for IPF, and approximately two-thirds of patients die within five years after diagnosis. Patients are typically treated with anti-inflammatory agents; however, none have been clinically proven to improve survival or quality of life for patients with IPF.

### SUMMARY OF THE INVENTION

One aspect of the present invention is a method of treating or preventing interstitial lung diseases, more specifically idiopathic pulmonary fibrosis, connective tissue or autoimmune disease-related pulmonary fibrosis, hypersensitivity pneumonitis, sarcoidosis, eosinophilic granuloma, also known as Langerhan's cell histiocytosis, chronic eosinophilic pneumonia, Wegener's granulomatosis, idiopathic pulmonary hemosiderosis; bronchiolitis obliterans, scleroderma, or lymphangioleiomyomatosis, comprising administering to a mammal, such as a human being, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof.

### DETAILED DESCRIPTION

The present invention relates to methods, uses, and compositions comprising (R)-N-(2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide, the structure of which is

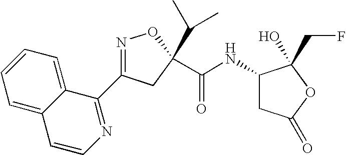

or a pharmaceutically acceptable salt thereof, a caspase inhibitor disclosed in WO 06/90997, herein incorporated by reference.

One aspect of the present invention is a method of treating or preventing interstitial lung disease comprising administering (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. In one embodiment, the method is the treating or preventing of idiopathic pulmonary fibrosis, connective tissue or autoimmune disease-related pulmonary fibrosis, hypersensitivity pneumonitis, sarcoidosis, eosinophilic granuloma, also known as Langerhan's cell histiocytosis, chronic eosinophilic pneumonia, Wegener's granulomatosis, idiopathic pulmonary hemosiderosis, bronchiolitis obliterans, scleroderma, or lymphangioleiomyomatosis.

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by aerosol delivery. In another embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by oral delivery.

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered in a daily dosage from 1 mg/kg to 500 mg/kg. In one embodiment, a daily dosage is administered from 10 mg/kg to 250 mg/kg. In one embodiment, a daily dosage is administered from 20 mg/kg to 200 mg/kg. In one embodiment, a daily dosage is administered in separate sub-doses, namely BID or TED.

In one embodiment, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof forms part of a combination with an additional therapeutic agent.

Another aspect of the present invention includes the use of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof in the manufacture of a medicament for the treatment or prevention of interstitial lung diseases.

Another aspect of the present invention includes a compound (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, for use in the treatment or prevention of interstitial lung diseases.

Another aspect of the present invention includes a pharmaceutical composition for the treatment of interstitial lung disease comprising (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof and one or more pharmaceutically acceptable carrier. In one embodiment, the pharmaceutical composition is for treating or preventing of idiopathic pulmonary fibrosis, connective tissue or autoimmune disease-related pulmonary fibrosis, hypersensitivity pneumonitis, sarcoidosis, eosinophilic granuloma, also known as Langerhan's cell histiocytosis, chronic eosinophilic pneumonia, Wegener's granulomatosis, idiopathic pulmonary hemosiderosis, bronchiolitis obliterans, scleroderma, or lymphangioleiomyomatosis.

In one embodiment, the composition is an aerosol formulation. In one embodiment, the composition is an oral formulation.

In one embodiment, the composition is provided in a daily dosage of from 1 mg/kg to 500 mg/kg, such as from 20 mg/kg to 200 mg/kg. The daily dosage may be one or more individual doses.

In one embodiment, the composition includes an additional therapeutic agent.

The scope of the present invention includes all combinations of aspects and embodiments.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

The pharmaceutical compositions of the present invention include administering (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, in the pure state or in the form of a composition in which the compounds are combined with any other pharmaceutically compatible product, which can be inert or physiologically active. The resulting pharmaceutical compositions can be used to prevent a condition or disorder in a subject susceptible to such a condition or disorder, and/or to treat a subject suffering from the condition or disorder. The pharmaceutical compositions described herein include one or more compounds of Formula 1 and/or pharmaceutically acceptable salts thereof, such as (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof.

The manner in which the compounds are administered can vary. The compositions may be administered orally, namely in liquid form within a solvent such as an aqueous or non-aqueous liquid, or within a solid carrier. Compositions for oral administration include pills, tablets, capsules, caplets, syrups, and solutions, including hard gelatin capsules and time-release capsules. Standard excipients include binders, fillers, colorants, solubilizers and the like. Compositions can be formulated in unit dose form, or in multiple or subunit doses. Preferred compositions are in liquid or semisolid form. Compositions including a liquid pharmaceutically inert carrier such as water or other pharmaceutically compatible liquids or semisolids can be used. The use of such liquids and semisolids is well known to those of skill in the art.

The compositions can also be administered via injection, i.e., intravenously, intramuscularly, subcutaneously, intraperitoneally, intraarterially, intrathecally; and intracerebroventricularly, Intravenous administration is the preferred method of injection. Suitable carriers for injection are well known to those of skill in the art and include 5% dextrose solutions, saline, and phosphate-buffered saline. The compounds can also be administered as an infusion or injection, namely, as a suspension or as an emulsion in a pharmaceutically acceptable liquid or mixture of liquids.

The compounds can also be administered directly to the respiratory tract by inhalation, namely, in the form of an aerosol either nasally or orally. Thus, one aspect of the present invention includes a novel, efficacious, safe, nonirritating, and physiologically compatible inhalable composition comprising (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. As stated herein, such a composition is suitable for treating idiopathic pulmonary fibrosis, scleroderma, or other interstitial lung diseases. Preferred pharmaceutically acceptable salts are inorganic acid salts including hydrochloride, hydrobromide, sulfate or phosphate salts, as they are known to cause less pulmonary irritation. Preferably, the inhalable formulation is delivered to the endobronchial space in an aerosol comprising particles with a mass median aerodynamic diameter (MMAD) between about 1 and about 5 μm. The (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof can be formulated for aerosol delivery using any device capable of producing particles with a mass median aerodynamic diameter (MMAD) between about 1 and about 5 μm. Common examples include nebulizers, pressurized metered dose inhalers (pMDIs), and dry powder inhalers (DPIs).

Non-limiting examples of nebulizers include atomizing, jet, ultrasonic, pressurized, vibrating porous plate or equivalent nebulizers. A jet nebulizer utilizes air pressure to break a liquid into aerosol droplets. An ultrasonic nebulizer works by a piezoelectric crystal that creates standing waves on the surface of the liquid that eject small aerosol droplets. A pressurized nebulization system forces solution under pressure through small pores to generate aerosol droplets via Rayleigh breakup. A vibrating porous plate device utilizes rapid vibration to pump liquid through the porous plate to generate appropriate droplet sizes via Rayleigh breakup.

Certain compositions of the invention described above provide the drug formulated in a solution permitting delivery of a therapeutically efficient amount of the drug by nebulization provided that the aerosol generated by the nebulization meets criteria required for efficient delivery. Therefore, the nebulizer which aerosolizes the formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof becomes an aspect of the invention. Only certain formulations can be efficiently nebulized using a given device, as the devices are sensitive to the physical and chemical properties of the formulation. Typically, the formulations which can be efficiently nebulized must contain small amounts of the compound, which are delivered in small volumes and conform to certain ranges of pH and osmolality.

For delivery by nebulizer, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is preferably dissolved in a minimal volume of about 0.5 to about 7 mL of an aqueous solvent having a pH between about 4.5 and about 7.5 and comprising chloride, bromine or iodine ions. Alternatively, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is not soluble in solvents and is formulated as a suspension. In one embodiment, the formulation has a shelf-life between about one and about two years. In another embodiment, the aqueous formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is prepared just prior to administration to assure the stability of the compound of and to assure a commercially acceptable shelf life of the drug.

In one embodiment, the formulation for nebulization is delivered to the endobronchial space in an aerosol comprising particles with a MMAD predominantly between about 1 μm and about 5 μm using a nebulizer able to aerosolize the formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof into particles of the required MMAD. One embodiment seeks optimal therapeutic effectiveness and avoidance of upper respiratory and systemic side effects by providing that the majority of aerosolized particles should not have a MMAD greater than about 5 μm. If an aerosol contains a large number of particles with a MMAD larger than 5 μm the particles tend to be deposited in the mouth and throat decreasing the amount of drug delivered to the site of inflammation and bronchoconstriction in the lower respiratory tract. If the MMAD of the aerosol is smaller than about 1 μm, then the particles have a tendency to remain suspended in the inhaled air and are subsequently exhaled during expiration.

In one embodiment, the solution or diluent used for preparation of the aerosol formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof has a pH range from about 3.0 to about 7.5, more preferably between about 3.5 and about 7.0. When the pH of the aerosol formulation is too acidic or too basic, it can cause bronchospasm and cough. Any aerosol having pH greater than 7.5 is to be avoided as the body tissues are unable to buffer alkaline aerosols. Aerosols with controlled pH below 3.0 and over 7.5 result in lung irritiation accompanied by severe bronchospasm cough and inflammatory reactions. In addition, aqueous formulations outside this pH range may contribute to more rapid degradation of the active ingredient. Consequently, in one embodiment, the aerosol formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is adjusted to a pH between about 3.0 and about 7.5 with a more preferred pH range from about 3.5 to about 7.0.

The solution for the aerosol formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof may require adjustment of the osmolality of the aerosol formulation to emulate the physiological conditions found in the healthy lungs. Bronchospasm or cough reflexes may not be totally repressed at the osmolality of the diluent for aerosolization, however, they can be sufficiently controlled and/or suppressed when the osmolality of the diluent is in a certain range. The given osmolality controls bronchospasm and the chloride concentration, as a permeant anion, controls cough. In one embodiment, formulations for nebulization of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof will have an osmolality between about 50 and about 1200 mOsm/kg, with reference to high osmolality solutions. Certain amounts of anion, such as chloride ion, may need to be added for successful and efficacious delivery of aerosolized (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. The chloride anion can be substituted with bromine or iodine anions, since both are permeant anions. In another embodiment, bicarbonate may be wholly or partially substituted for chloride ion.

When formulated and delivered according to the method of the invention, the aerosol formulation for nebulization delivers a therapeutically efficacious dose of the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof to the lungs. The amount of drug administered must be adjusted to reflect the efficiency of the delivery of a therapeutically efficacious dose of the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof. In a preferred embodiment, a combination of the aqueous aerosol formulation with the atomizing, jet, pressurized, vibrating porous plate, or ultrasonic nebulizer permits, depending on the nebulizer, about, at least, 20, to about 90%, typically about 70% delivery of the administered dose of the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof into the airways. In a preferred embodiment, at least about 30 to about 50% of the active compound is delivered. More preferably, about 70 to about 90% of the active compound is delivered.

In another embodiment, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is delivered as a dry inhalable powder. The (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered endobronchially as a dry powder formulation to efficaciously deliver fine particles of compound into the endobronchial space using dry powder or metered dose inhalers. For delivery by DPI, the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is processed into particles with, predominantly, MMAD between about 1 μm and about 5 μm by milling, spray drying, critical fluid processing, or precipitation from solution. Media milling, jet milling and spray-drying devices and procedures capable of producing the particle sizes with a MMAD between about 1 μm and about 5 μm are well known in the art. In one embodiment, excipients are added to the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof before processing into particles of the required sizes. In another embodiment, excipients are blended with particles of the required size to aid in dispersion of the drug particles, for example by using lactose as an excipient.

Particle size determinations are made using devices known in the art. Examples of such devices include a multi-stage Anderson cascade impactor or other suitable method such as those specifically cited within the US Pharmacopoeia Chapter 601 as characterizing devices for aerosols within metered-dose and dry powder inhalers.

In one embodiment, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is delivered as a dry powder using a device such as a dry powder inhaler or other dry powder dispersion devices. Non-limiting examples of dry powder inhalers and devices include those disclosed in U.S. Pat. No. 5,458,135; U.S. Pat. No. 5,740,794; U.S. Pat. No. 5,775,320; U.S. Pat. No. 5,785,049; U.S. Pat. No. 3,906,950; U.S. Pat. No. 4,013,075; U.S. Pat. No. 4,069,819; U.S. Pat. No. 4,995,385; U.S. Pat. No. 5,522,385; U.S. Pat. No. 4,668,218; U.S. Pat. No. 4,667,668; U.S. Pat. No. 4,805,811; and U.S. Pat. No. 5,388,572. One design is a metering device in which a reservoir for the drug is placed within the device and the patient adds a dose of the drug into the inhalation chamber. Another design is a factory-metered device in which each individual dose has been manufactured in a separate container. Both systems depend on the formulation of the drug into small particles of MMAD from 1 μm and about 5 μm and often involve co-formulation with larger excipient particles such as, but not limited to, lactose. Drug powder is placed in the inhalation chamber (either by device metering or by breakage of a factory-metered dosage) and the inspiratory flow of the patient accelerates the powder out of the device and into the oral cavity. Non-laminar flow characteristics of the powder path cause the excipient-drug aggregates to decompose, and the mass of the large excipient particles causes their impaction at the back of the throat, while the smaller drug particles are deposited deep in the lungs. In certain embodiments, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is delivered as a dry powder using either type of dry powder inhaler as described herein, wherein the MMAD of the dry powder, exclusive of any excipients, is predominantly in the range of 1 μm to about 5 μm. Further, in certain embodiments, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, is delivered with an active inhaler, such as that sold under the trade name MicroDose, with reference to U.S. Pat. No. 7,334,577. As is appreciated by those skilled in the art, there are devices that use means other than turbulence to effectuate flow, such as beads, with reference to U.S. Pat. No. 6,427,688, or other similar means. (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof may be used in such devices.

In one embodiment, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is delivered as a dry powder or as a solution using a metered dose inhaler. Non-limiting examples of metered dose inhalers and devices include those disclosed in U.S. Pat. No. 5,261,538; U.S. Pat. No. 5,544,647; U.S. Pat. No. 5,622,163; U.S. Pat. No. 4,955,371; U.S. Pat. No. 3,565,070; U.S. Pat. No. 3,361,306; and U.S. Pat. No. 6,116,234. In certain embodiments, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is delivered as a dry powder using a metered dose inhaler wherein the MMAD of the dry powder, exclusive of any excipients, is predominantly in the range of about 1-5 μm.

The amount of active ingredient that may be combined with the excipients to produce a single dosage form that will vary depending upon the host treated and the particular mode of administration. (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is dosed in a therapeutically effective amount ranging from about 10 to about 5000 μg. The dose will be determined by the host treated and the severity of the disease as determined by those physicians skilled in the art. Preferably, the drug will be administered four, three, two, or most preferably once a day.

In another aspect of the invention, a combination of an aerosol formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof and a device significantly enhances the efficiency and speed of drug administration. Currently, for example, the average time for administration of other aerosolized drugs, such as for example tobramycin, is 15-20 minutes per dose. The time required for this treatment represents a significant burden to the patient and contributes to reduced compliance with the recommended dosage regimen.

In a preferred embodiment, the aerosolizable formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, is delivered by a device capable of delivering a therapeutically effective dose in less than 15 minutes, more preferably in less than 10 minutes, and most preferably in less than 5 minutes. Non-limiting examples of these devices include those disclosed in U.S. Pat. No. 6,962,151.

Further aerosol formulations exist, including those directed to the microparticles that carry the active ingredient such as U.S. patent application Ser. No. 11/189,553, published as US 2006/00147520, herein incorporated by reference. The scope of the present invention is intended to cover any aerosol formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof.

The compounds can also be administered transdermally, such as through use of a transdermal patch or iontophoretically, or by sublingual or buccal administration.

Although it is possible to administer the compounds in the form of a bulk active chemical, it is preferred to present each compound in the form of a pharmaceutical composition or formulation for efficient and effective administration. Exemplary methods for administering such compounds will be apparent to the skilled artisan. The usefulness of these formulations can depend on the particular composition used and the particular subject receiving the treatment. These formulations can contain a liquid carrier that can be oily, aqueous, emulsified or contain certain solvents suitable to the mode of administration.

The compositions can be administered intermittently or at a gradual, continuous, constant or controlled rate to a warm-blooded animal (e.g., a mammal such as a mouse, rat, cat, rabbit, dog, pig, cow, or monkey), but advantageously are administered to a human being. In addition, the time of day and the number of times per day that the pharmaceutical formulation is administered can vary.

In an embodiment of the present invention and as will be appreciated by those skilled in the art, the compound of the present invention may be administered in combination with other therapeutic compounds. For example, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof can be used in combination with PPARγ agonists, PI3 Kinase inhibitors, MAP kinase inhibitors (such as p38 kinase inhibitors), matrix metalloproteinase inhibitors, serine protease inhibitors steroids, β₂-adrenergic receptor agonists, M3 muscarinic receptor antagonists, PDE4 inhibitors, inhibitors of products of 5-lipoxygenase pathway, inhibitors of products of cyclooxygenase pathway, inhibitors of TGFβ signaling pathway, inhibitors of pro-inflammatory or pro-fibrotic chemokines, and cytokines.

Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof in combination with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof may be used in the treatment of a variety of disorders and conditions and, as such, may be used in combination with a variety of other suitable therapeutic agents useful in the treatment or prophylaxis of those disorders or conditions.

The following examples are provided to illustrate the present invention, and should not be construed as limiting thereof. In these examples, all parts and percentages are by weight, unless otherwise noted.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. As used herein, such an amount of (R)-N-((2S,3S)-2-(fluoromethyl)-2-hydroxy-5-oxo-tetrahydrofuran-3-yl)-5-isopropyl-3-(isoquinolin-1-yl-4,5-dihydroisoxazole-5-carboxamide or a pharmaceutically acceptable salt thereof present in a composition that is needed to provide a desired level of drug in the secretions and tissues of the airways and lungs, or alternatively, in the bloodstream of a subject to be treated to give an anticipated physiological response or desired biological effect when such a composition is administered by inhalation. The precise amount will depend upon numerous factors, for example the particular formulation of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof, the specific activity of the composition, the delivery device employed, the physical characteristics of the composition, its intended use, as well as patient considerations such as severity of the disease state, patient cooperation, etc., and can be determined by one skilled in the art based upon the information provided herein.

Typically, the effective dose of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof generally requires administering the compound in an amount of less than 500 mg/kg of patient weight. The effective doses typically represent that amount administered as a single dose, or as one or more doses administered over a 24-hour period.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. See Hoyer, D. and Boddeke, H., Trends Pharmacol. Sci. 14(7): 270-5 (1993), herein incorporated by reference with regard to such teaching. Where such contextually specific evaluations are relevant, and how they might be relevant in the context of the present invention, will be apparent to one of ordinary skill in the art.

As used herein, the terms “prevention” or “prophylaxis” include any degree of reducing the progression of or delaying the onset of a disease, disorder, or condition. The term includes providing protective effects against a particular disease, disorder, or condition as well as amelioration of the recurrence of the disease, disorder, or condition. Thus, in another aspect, the invention provides a method for treating a subject having or at risk of developing or experiencing a recurrence of a disorder mediated through caspase inhibition. The compounds and pharmaceutical compositions of the invention may be used to achieve a beneficial therapeutic or prophylactic effect, for example, in a subject with a need therefor.

As noted, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide is a caspase inhibitor disclosed in WO 06/90997, herein incorporated by reference. (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide may be made by a variety of methods. One illustrative synthetic method is set out below. In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3rd Edition, John Wiley & Sons, incorporated by reference with regard to protecting groups), These groups are removed at a convenient stage of the compound synthesis using methods that are readily apparent to those skilled in the art. The selection of processes as well as the reaction conditions and order of their execution shall be consistent with the preparation of compounds of the present invention.

The compounds can be prepared according to the methods described below using readily available starting materials and reagents. In these reactions, variants may be employed which are themselves known to those of ordinary skill in this art, but are not mentioned in greater detail.

### Synthetic Examples

As referenced in WO 06190997, a general preparation for compounds of Formula 1

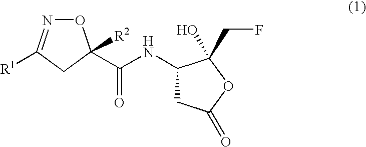

including (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide provides:
(a) activating a compound of following formula (2),

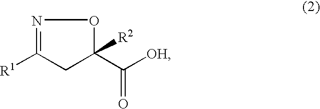

then reacting it with a compound of the following formula (4),

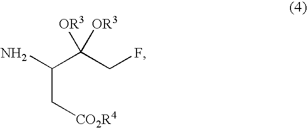

to produce a compound of the following formula (13),

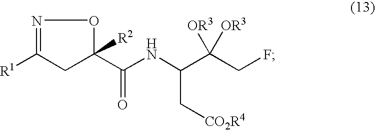

(b) hydrolyzing the compound of the following formula (13) to produce a compound of the following formula (14),

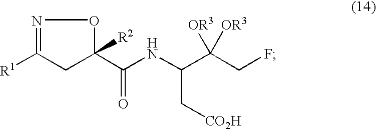

(c) deprotecting the compound of the following formula (14); and (d) carrying out crystallization-induced dynamic transformation. In the above-referenced general process, R¹ is alkyl or aryl; R² is alkyl, each R³ individually is alkyl, or both R³ together with oxygen atom to which they are attached form a heterocycle, and R⁴ is alkyl.

One embodiment provides an activation reagent to activate the compound of formula (2), selected from the group consisting of oxalyl chloride, trimethylacetyl chloride, phosphoryl tri-chloride, and thionyl chloride. Further, step (a) preferably is carried out in the presence of base selected from the group consisting of triethylamine, tri(n-butyl)amine, diisopropy-lethylamine, pyridine, 4-dimethylaminopyridine and 4-(4-methyl-piperidine-1-yl)-pyridine. One preferable ratio provides that the base is used in an amount of 1.0 to 10.0 equivalents to the compound of formula (2). Preferably, the reaction in the step (a) is carried out in one or more solvents selected from the group consisting of dichloromethane, chloroform, tetrahydrofuran, dimethoxyethane, dioxane, and ethyl acetate.

One embodiment provides that the compound of formula (4) in step (a) is used in an amount of 1.0 to 3.0 equivalents to the compound of formula (2). The hydrolysis in step (b) preferably is carried out in the presence of base selected from the group consisting of lithium hydroxide, preferably either anhydrous or monohydrate crystalline, sodium hydroxide, potassium hydroxide, and calcium hydroxide. In one embodiment, the base is used in an amount of 0.1 to 10.0 equivalents to the compound of formula (13).

Preferably, the reaction in the step (b) is carried out in one or more solvents selected from the group consisting of methanol, ethanol, n-propanol, isopropanol, tetrahydrofuran, dimethoxyethane, dioxane, and dichloromethane, or in a mixed solvent including one or more of the solvents selected from the above group and water.

Preferably, the deprotection reaction in the step (c) is carried out in the presence of acid, such as hydrochloric acid, sulfuric acid, or trifluoroacetic acid, and it is preferable that the acid is used in an amount of 0.1 to 20.0 equivalents to the compound of formula (14).

Preferably, the deprotection reaction in the step (c) is carried out in the presence or absence of solvent. If conducted in the presence of solvent, the solvent preferably is selected from dichloromethane or chloroform.

The crystallization-induced dynamic transformation reaction in the step (d) can be carried out by adding the compound of formula (1) as seed, or carried out in the presence of seed and a catalytic amount of base, wherein the base is preferably an amine selected from the group consisting of triethylamine, tri(n-butyl)amine, diisopropylethylamine, diisopropylamine, pyridine, 4-dimethylaminopyridine, 4-(4-methyl-piperidine-1-yl)-pyridine, optically active 1-phenylethylamine, and optically active 1-naphthylethylamine.

In the step (d), it is preferable to use said amine in an amount of 0.001 to 1.0 equivalent to the compound of formula (14), and more preferable to use 0.03 to 0.5 equivalent. If the amount of used amine is too little, the reaction rate becomes slower, and if the amount is too much, the yield of the compound of formula (1) is decreased.

Further, it is preferable that the crystallization-induced dynamic transformation reaction in the step (d) is carried out in one or more solvents selected from the group consisting of toluene, benzene, dichlorobenzene, tetrahydrofuran, dimethoxyethane, dioxane, ethyl acetate, dichloromethane, acetonitrile, methyl t-butylether, and di-ethylether.

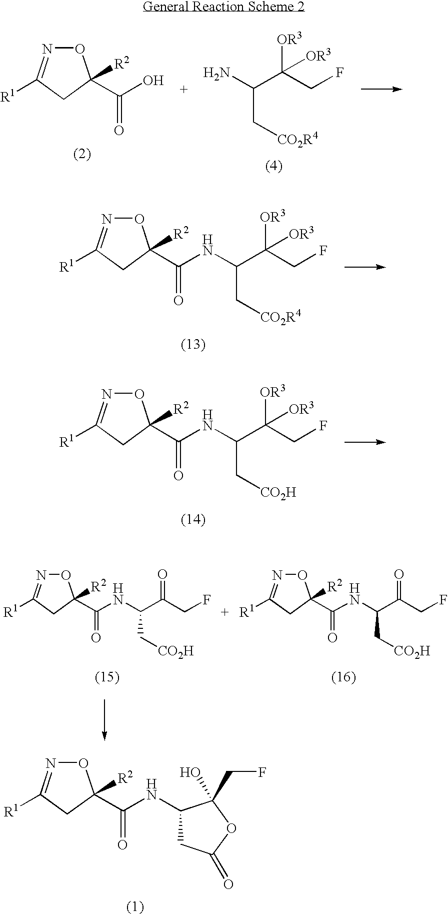

The isoxazoline derivative of formula (2) having high optical activity is prepared according to the process disclosed in PCT/KR2004/02139 filed on Aug. 17, 2004, herein incorporated by reference, and then combined with the compound of formula (4) to

produce the compound of formula (13). Then, the compound of formula (13) is ester-hydrolyzed to produce the compound of formula (14), and the deprotection reaction of the ketal moiety of the compound of formula (14) is carried out to obtain a mixture of the compounds of formula (15) and formula (16), which is effectively transformed into the compound of formula (1) by selective dynamic crystallization.

In particular, if the mixture of the compounds of formula (15) and formula (16) is dissolved in organic solvent, and the seed of the compound of formula (1) is added to the solution, only the compound of formula (15) in the mixture is transformed into the compound of formula (1) to be isolated as solid.

Also, if the mixture of the compounds of formula (15) and formula (16) is treated with a catalytic amount of base together with seed, both the compound of formula (15) and the compound of formula (16) are transformed into the compound of formula (1), to produce the compound of formula (1) with higher yield, with reference to General Reaction Scheme 3.

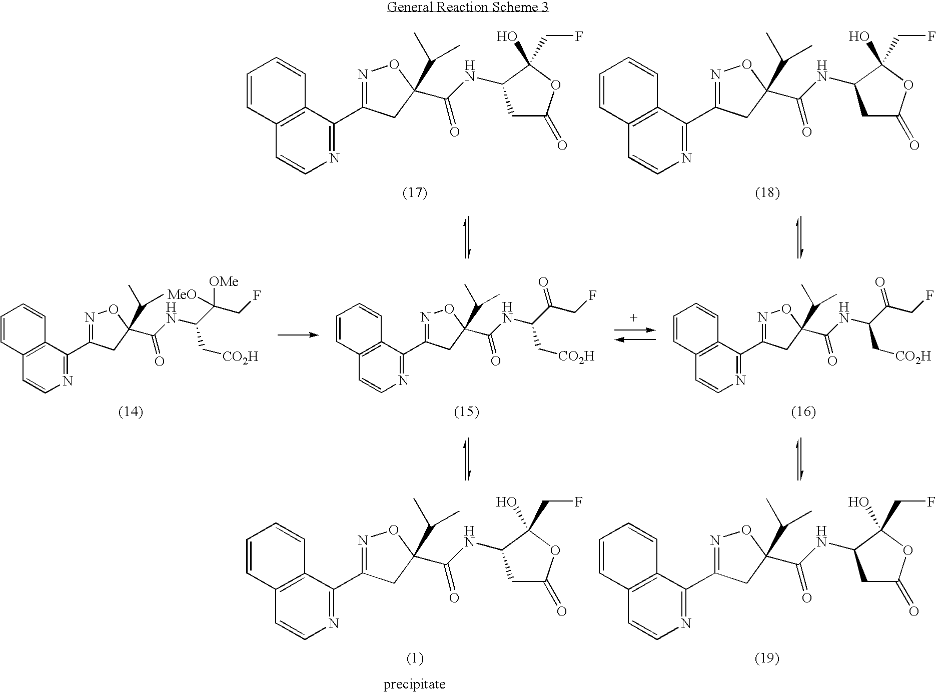

The compound of formula (15) is in equilibrium with the compound of formula (16) due to the base present in solution. Also, the compound of formula (15) is in equilibrium with the compounds of formula (17) and formula (1), and the compound of formula (16) is in equilibrium with the compounds of formula (18) and formula (19). Among them, the compound of formula (1) having good crystallizing property selectively precipitates, and so the equilibrium of all the compounds moves to the compound of formula (1), thereby selectively giving only the compound of formula (1) with high yield from the mixture of the compounds of formula (15) and formula (16).

### Preparation Example 1

### 1-Fluoro-4-trimethylsilanyl-3-butyn-2-one

49.1 g (499 mmol) of trimethylsilylacetylene was dissolved in 250 mL of anhydrous tetrahydrofuran, and the inner temperature was lowered to about −55° C., and then 210 mL (525 mmol) of 2.5 M n-BuLi in n-hexane was added thereto over about 25 minutes with maintaining the inner temperature below −30° C. After stirring for about 40 minutes, 52.9 g (499 mmol) of ethyl fluoroacetate was added to the reaction mixture over 5 minutes with maintaining the inner temperature below −25° C., and then 74.4 g (524 mmol) of BF₃-OEt was added thereto over 15 minutes with maintaining the inner temperature −55° C. to −65° C. After finishing the addition, the reaction mixture was stirred at 20° C. for 2 hours, and 250 mL of 10% ammonium chloride aqueous solution was added thereto to finish the reaction. The organic layer was separated, and the aqueous layer was extracted with 200 mL of ethylacetate. The combined organic phase was washed with 250 mL of brine, and concentrated under reduced pressure. The residue was distilled under vacuum at 10 mbar and 68° C. to give 1-fluoro-4-trimethylsilanyl-3-butyn-2-one

(67.3 g, 85%) as clear oil.

¹H NMR (500 MHz, CDCl3): 4.90 (d, J=47.1 Hz, 2H), 0.26 (s, 9H)

¹³C NMR (125 MHz, CDCl3): 181.0 (d, J=21.5 Hz), 104.0, 98.1, 84.8 (d, J=187 Hz)

### Preparation Example 2

### 4-Fluoro-3,3-dimethoxy-1-butyne

33.6 g (316 mmol) of trimethyl orthoformate and 6.0 g (31.5 mmol) of p-TsOH—H₂O together with 50.0 g (316 mmol) of 1-fluoro-4-trimethylsilanyl-3-butyn-2-one obtained from the Preparation Example 1 were put into 260 mL of methanol, and stirred at reflux temperature (inner temperature 60˜64° C.) for about 6 hours. The reaction mixture was concentrated under reduced pressure to remove about 130 ml. of solvent, and was diluted with 260 mL of methylene chloride. 130 mL of 10% aqueous sodium hydrogen carbonate solution was added thereto and layer-separated, and the water layer was extracted by using 130 mL of methylene chloride. The combined organic layer was concentrated under reduced pressure to give 4-fluoro-3,3-dimethoxy-1-trimethylsilylbutyne (59.0 g, 92%) as an intermediate, a precursor compound of the object compound 4-fluoro-3,3-dimethoxy-1-butyne. This compound was used in the next reaction without further purification.

¹H NMR (500 MHz, CDCl3): 4.38 (d, J=47.1 Hz, 2H), 3.40 (s, 6H), 0.20 (s, 9H)

59.0 g (289 mmol) of 4-fluoro-3,3-dimethoxy-1-trimethylsilylbutyne, a precursor compound of 4-fluoro-3,3-dimethoxy-1-butyne obtained from the above, was dissolved in 280 mL of methylene chloride, treated with 59 mg (0.183 mmol) of tetra-n-butylammoniumbromide and 347 mL (347 mmol) of 1 N sodium hydroxide aqueous solution, and stirred for about 2 hours. The organic layer was separated, and the aqueous layer was extracted with 110 mL of methylene chloride. The combined organic layer was washed with 110 mL of brine, and concentrated under reduced pressure to give the object compound 4-fluoro-3,3-dimethoxy-1-butyne (40.9 g, quantitative yield). This compound was used in the next reaction without further purification.

¹H NMR (500 MHz, CDCl3): 4.42 (d, J=47.1 Hz, 2H), 3.42 (s, 6H), 2.64 (s, IH)

¹³C NMR (125 MHz, CDCl3): 96.1 (d, J=20.3 Hz), 82.9 (d, J=180 Hz), 77.5, 75.5, 51.0

### Preparation Example 3

### Ethyl 5-fluoro-4,4-dimethoxy-2-pentynoate

A solution of 40.9 g (405 mmol) of diisopropylamine in 270 mL of tetrahydrofuran was cooled to 0° C., and 112 g (405 mmol) of 2.5 M n-BuLi in n-hexane was added thereto over about 1 hour with maintaining the inner temperature below 14° C. The reaction mixture was stirred at 0° C. for about 30 minutes, and the temperature was adjusted to −78° C. A solution of 41.0 g (311 mmol) of the compound 4-fluoro-3,3-dimethoxy-1-butyne obtained from the above Preparation Example 2 dissolved in 160 mL of tetrahydrofuran was added to the reaction mixture over about 2 hours with maintaining the inner temperature below −40° C., and then 60.4 g (557 mmol) of ethyl chloroformate was added thereto over about 1 hour with maintaining the inner temperature below −40° C., and further the reaction mixture was stirred at 0° C. for about 2 hours. 250 mL of 10% ammonium chloride aqueous solution was added to the reaction mixture to finish the reaction, and the organic layer was separated. The aqueous layer was extracted with 100 mL of ethyl acetate, and the combined organic layer was washed with 100 mL of brine and concentrated under reduced pressure to give the crude object compound ethyl 5-fluoro-4,4-dimethoxy-2-pentynoate (95.0 g, calculated yield 70%). This compound was used in the next reaction without further purification

¹/H NMR (500 MHz, CDCl3): 4.45 (d, J=46.5 Hz, 2H), 4.25 (q, J=7.1 Hz, 2H), 3.43 (s, 6H), 1.31 (t, J=7.3 Hz, 3H)

### Preparation Example 4

### Ethyl 3-(benzylamino)-5-fluoro-4,4-dimethoxypentanoate

88 g (431 mmol) of the crude compound ethyl 5-fluoro-4,4-dimethoxy-2-pentynoate obtained from the above Preparation Example 3 was dissolved in 430 mL of methyl-t-butyl ether (MTBE), and the temperature was adjusted to 0° C. 31.4 g (293 mmol) of benzylamine was added to the reaction mixture, stirred at 20° C. for about 1 hour, and diluted with 450 mL of methyl-t-butyl ether. Again, the temperature of the reaction mixture was adjusted to 0° C., 33 g (873 mmol) of NaBH₄ was added to the reaction mixture, and then 259 g (4320 mmol) of acetic acid was added thereto over about 30 minutes. The reaction mixture was maintained at 0° C., and 880 mL (2640 mmol) of 3 N sodium hydroxide aqueous solution was slowly added thereto over about 2 hours. The organic layer was separated, and the separated organic layer was washed with 880 mL of 10% ammonium chloride aqueous solution, and then 880 mL of 1 N hydrochloric acid aqueous solution was added thereto. The aqueous layer was separated, washed with 400 mL of methyl-t-butyl ether, and basified by using 246 mL of 10 N sodium hydroxide aqueous solution, and extracted with 700 mL×2 of methyl-t-butyl ether. The combined organic layer was washed with 400 mL of brine, and concentrated under reduced pressure to give the object compound ethyl 3-(benzylamino)-5-fluoro-4,4-dimethoxypentanoate [60.0 g, 44%]. This compound was used in the next reaction without further purification

¹H NMR (400 MHz, CDCl3): 7.35-7.21 (m, 5H), 4.53 (2 dd, J=46.8, 10.4 Hz, 2H), 4.13 (q, J=7.2 Hz, 2H), 3.80 (2d, J=12.8 Hz, 2H), 3.53 (dd, J=8.4, 4.0 Hz, IH), 3.30 (s, 3H), 3.22 (s, 3H), 2.79 (dd, J=15.6, 3.6 Hz, IH), 2.40 (ddd, J=15.6, 8.0, 1.6 Hz, IH), 1.25 (t, J=7.2 Hz, 3H)

### Preparation Example 5

### Ethyl 3-amino-5-fluoro-4,4-dimethoxypentanoate

18.3 g (58.5 mmol) of the compound ethyl 3-(benzylamino)-5-fluoro-4,4-dimethoxypentanoate obtained from the above Preparation Example 4 was dissolved in 180 mL of ethanol, and debenzylation was carried out by using activated carbon 5% palladium catalyst (5% Pd/C) at the hydrogen pressure of 50 psi for about 4 hours. The reaction mixture was filtered through 5.0 g of Cellite pad, and washed with 90 mL of ethanol, and the filtrate was concentrated under reduced pressure to give the object compound ethyl 3-amino-5-fluoro-4,4-dimethoxypentanoate (12.8 g, 98%). This compound was used in the next step without any purification.

¹H NMR (500 MHz, CDCl3): 4.53 (2 dd, J=46.5, 10.4 Hz, 2H), 4.14 (q, J=7.3 Hz, 2H), 3.57 (dd, J=11.0, 1.9 Hz, IH), 3.29 (d, J=11.7 Hz, 6H), 2.73 (dd, J=16.5, 2.5 Hz, IH), 2.36 (ddd, J=16.5, 10.4, 2.5 Hz, IH), 1.25 (t, J=7.3 Hz, 3H)

### Preparation Example 6

### 5-fluoro-3-[((R)-5-isopropyl-3-(I-isoquinolinyl)-4,5-dihydro-isoxazole-5-carbonyl)-amino]-4,4-dimethoxy-pentanoic acid ethyl ester

15.5 g (54.5 mmol) of (5R)-5-isopropyl-3-(1-isoquinolinyl)-4,5-dihydro-5-isoxazole carboxylic acid was dissolved in 150 mL of methylene chloride, the temperature was adjusted to 0° C., and then 7.1 mL (81.7 mmol) of oxalyl chloride and 0.2 mL (2.6 mmol) of DMF were added thereto with maintaining the inner temperature below 12° C. The reaction mixture was stirred at 20° C. for about 2 hours, and concentrated under reduced pressure. The reaction mixture was dissolved in 150 mL of methylene chloride, the temperature was adjusted to 0° C., triethylamine was added thereto, and a solution of 12.8 g (57.4 mmol) of the compound ethyl 3-amino-5-fluoro-4,4-dimethoxypentanoate obtained from Preparation Example 5 dissolved in 30 mL of methylene chloride was slowly added thereto over 20 minutes. The reaction mixture was stirred at 25° C. for 1.5 hours, a mixed solution of 120 mL of 10% sodium hydrogen carbonate aqueous solution and 60 mL of 1 N sodium hydroxide aqueous solution was added thereto to finish the reaction. The organic layer was separated, and the aqueous layer was extracted with 150 mL×3 of methylene chloride. The combined organic layer was concentrated under reduced pressure to give the object compound 5-fluoro-3-[(((R)-5-isopropyl-3-(1-isoquinolinyl)-4,5-dihydro-isoxazole-5-carbonyl)-amino]-4,4-dimethoxy-pentanoic acid ethyl ester (30.1 g, quantitative yield). This compound was used in the next step without any purification.

¹H NMR (500 MHz, CDCl₃): 9.12 (q, 1H), 8.53 (m, 1H), 7.85-7.25 (m, 4H), 4.80 (m, 1H), 4.54-4.34 (m, 2H), 4.14 (q, J=7.4 Hz, 2H), 3.99 (2d, J=18.4 Hz, 1H), 3.81 (m, 1H), 3.78 (2d, J=8.6 Hz, 1H), 3.33 (d, 3H), 3.20 (d, 3H), 2.75 (m, 3H), 2.53 (m, 1H), 2.39 (heptet, J=6.7 Hz, 1H), 1.27 (t, J=7.4 Hz, 1.5H), 1.07 (m, 6H), 0.97 (t, J=7.4 Hz, 1.5H)

### Preparation Example 7

### 5-Fluoro-3-[((R)-5-isopropyl-3-(1-isoquinolinyl)-4,5-dihydro-isoxazole-5-carbonyl)-amino]-4,4-dimethoxy-pentanoic acid

30.1 g (61.6 mmol) of the compound 5-fluoro-3-[((R)-5-isopropyl-3-(1-isoquinolinyl)-4,5-dihydro-isoxazole-5-carbonyl)-amino]-4,4-dimethoxy-pentanoic acid ethyl ester obtained from the above Preparation Example 6 together with 7.76 g (185 mmol) of lithium hydroxide monohydrate were dissolved in a mixed solvent of 168 mL of tetrahydrofuran and 42 mL of water, and stirred at about 40° C. for 4 hours. The reaction mixture was concentrated under reduced pressure to remove tetrahydrofuran in the solvent, 180 mL of 1 N sodium hydroxide aqueous solution was added thereto, and the mixture was washed with 120 mL×2 of toluene. The aqueous layer was acidified with 66 mL of 6 N hydrochloric acid aqueous solution, and extracted with 150 mL×3 of methylene chloride, and the combined organic layer was concentrated under reduced pressure to give the object compound 5-fluoro-3-[((R)-5-isopropyl-3-(1-isoquinolinyl)-4,5-dihydro-isoxazole-5-carbonyl)-amino]-4,4-dimethoxy-pentanoic acid (25.4 g, 89%). This compound was used in the next step without any purification.

¹H NMR (400 MHz, CDCl₃): 9.10-8.92 (m, 1H), 8.52 (m, 1H), 7.86-7.13 (m, 4H), 4.77 (m, 1H), 4.54-4.34 (m, 2H), 3.95 (2d, J=8.0 Hz, 1H), 3.75 (2d, J=18.4 Hz, 1H), 3.35-3.16 (2d, 6H), 2.78 (2 dd, J=16.0, 4.4 Hz 1H), 2.54 (m, 1H), 2.39 (m, 1H), 2.35 (s, 1H), 1.06 (m, 6H)

### Example 1

### (45,SS)-5-fluoromethyl-5-hydroxy-4-({[(5R)-5-isopropyl-3-(I-isoquinolinyl)-4,5-dihydro-5-isoxazolyl]carbonyl}amino)-2-dihydrofuranone

17.0 g (36.9 mmol) of the compound 5-fluoro-3-[((R)-5-isopropyl-3-(I-isoquinolinyl)-4,5-dihydro-isoxazole-5-carbonyl)-amino]-4,4-dimethoxy-pentanoic acid obtained from the above Preparation Example 7 and 6.6 mL (110 mmol) of acetic acid were dissolved in 123 mL (738 mmol) of 6 N hydrochloric acid aqueous solution, and stirred for about 4 hours. The inner temperature of the reaction mixture was adjusted to 0° C., and 150 mL of ethyl acetate was added thereto. 220 mL (660 mmol) of 3 N sodium hydroxide aqueous solution was slowly added to adjust the pH to about 3. The organic layer was separated, and the aqueous layer was extracted with 150 mL×2 of ethyl acetate. The combined organic phase was washed with 100 mL of brine, and concentrated under reduced pressure. The residue was diluted with 50 mL of toluene, and concentrated again under reduced pressure to give a mixture of the compounds of formula (15) and formula (16) as above referenced (15.4 g, quantitative yield, chemical purity: 87.0%).

¹H NMR (500 MHz, DMSO-δ6): 8.99 (m, 1H), 8.65 (m, 1H), 8.19-7.78 (m, 4H), 5.15 (m, 1.5H), 4.77 (m, 1H), 4.42 (m, 0.5H), 3.91 (2d, J=17.6 Hz, 1H), 3.74 (m, 1H), 2.99 (m, 0.2H), 2.82 (m, 1H), 2.63 (m, 0.8H), 2.33 (m, 1H), 0.97 (m, 6H)

To 146 mL of toluene was added 14.6 g (35.2 mmol) of the mixture of the compounds of formula (15) and formula (16) (chemical purity: 87.0%), and the mixture was heated up to 100° C. to dissolve it completely. Then, 14 mg of seed of the object compound was added thereto, the temperature was slowly lowered to 20° C., and the reaction mixture was stirred to produce solid. 0.25 mL (1.8 mmol) of diisopropylamine was added thereto, and stirred at 20° C. for about 2 weeks, to confirm the ratio between the compound of formula (15) and the compound of formula (16)—92.8:7.2 by HPLC. The reaction mixture was concentrated under reduced pressure to remove toluene, 88 mL of ethyl acetate was added thereto, and the mixture was heated up to 65° C. to dissolve it completely. Then, 88 mL of normal hexane was added thereto, and the temperature was slowly lowered and stirred at about 20° C. for 2 days. The resulting solid was filtered, and washed with a mixed solution of 15 mL of ethyl acetate and 15 ml of normal hexane. After drying the solid with nitrogen, the object compound, a white solid was obtained in 54.7% of yield (8.0 g, chemical purity 98.6%). Solid NMR data of the crystalline form was obtained by using VACP MAS (variable amplitude cross polarization magic angle spinning) at 9 kH spinning rate.

¹H NMR (CDCl₃): 9.02 (bs, 1H), 8.54 (d, J=5.5 Hz, 1H), 7.85 (d, J=7.95 Hz, 1H), 7.70 (m, 3H), 7.60 (bs, 1H), 4.86 (bs, 1H), 4.2-5.2 (bs, 2H), 4.05 (b, J=19.0 Hz, 1H), 3.78 (b, J=19.0 Hz, 1H), 2.7-3.1 (bm, 2H), 2.40 (m, 1H), 1.08 (dd, J=6.7, 4.9 Hz, 6H);

¹³C NMR (CDCl₃): 173.8, 172.4, 160.2, 147.6, 141.7, 136.8, 130.7, 129.0, 127.4, 127.3, 126.8, 122.9, 92.3, 82.7 (d, J=215 Hz), 48.9 (b), 44.6, 34.4, 33.9, 17.7, 16.3;

¹³C NMR (solid): 176.4, 171.8, 160.3, 150.2, 139.5, 137.5, 132.3 (2C), 127.7 (3C), 123.0, 104.3, 94.1, 86.4, 48.8, 42.9, 32.7 (2C), 19.6, 15.4;

Mass (ESI): 416.14 (M+I).

### Biological Example 1

### Oral Administration of (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide Inhibits Inflammation and Fibrosis Induced by Bleomycin in the Lungs of Rats

The bleomycin rat model was used in this study to evaluate (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide as a possible therapeutic drug for the treatment of idiopathic pulmonary fibrosis (IPF) and other fibrotic lung diseases. The bleomycin animal model of IPF is an art-recognized model for evaluating potentially therapeutic drugs for the treatment of IPF (see, e.g., F. Chua et al., Pulmonary Fibrosis, Searching for Model Answers, Am. J. Respir. Cell. Mol. Biol., 33:9-13 (2005); A. Moeller et al., The bleomycin animal model: A useful tool to investigate treatment options for idiopathic pulmonary fibrosis, International Journal of Biochemistry and Cell Biology, 40: 362-382 (2008)).

In this Example, (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide is referred to as the “test compound” for ease of reference. Table 1 shows the experimental design. The abbreviations used in Table 1 are Bleo (bleomycin), IT (intratracheal), mg/kg (milligram per kilogram), N (number) and n/a (not applicable).

TABLE 1

| | | | Test | |
|---|---|---|---|---|
| | | Bleo IT | Compound | |
| Exposure Group | N | (3.5 mg/kg) | Dose | Necropsy Day |
| Vehicle control | 8 | n/a | n/a | 14 |
| Vehicle control | 8 | n/a | n/a | 28 |
| Bleomycin control | 8 | Day 1 | n/a | 14 |
| Bleomycin control | 8 | Day 1 | n/a | 28 |
| Bleo plus test | 8 | Day 1 | Day 1-14 | 14 |
| compound (10 mg/kg) | | | | |
| Bleo plus test | 8 | Day 1 | Day 1-27 | 28 |
| compound (10 mg/kg) | | | | |
| Bleo plus test | 8 | Day 1 | Day 1-14 | 14 |
| compound (30 mg/kg) | | | | |
| Bleo plus test | 8 | Day 1 | Day 1-27 | 28 |
| compound (30 mg/kg) | | | | |

The rats receiving the test compound were gavaged twice a day from day zero (the day of bleomycin administration) through the day prior to necropsy. On days 14 and 28 the designated rats were euthanized with a pentobarbital-based solution, the chest cavity was opened and examined for gross pathology. The right lung was removed, canulated, and lavaged for determination of white blood cell counts. The lavaged tissue was flash frozen and analyzed for collagen content by measuring the amount of collagen. The left lung was fixed for histopathology. Homogenates from the lungs were analyzed for cytokines.

Intratracheal administration of bleomycin induces a two-stage response in the lungs of rats. The first stage is an inflammatory response which peaks at about 14 days after bleomycin administration, and the second stage is a fibrotic response that is apparent by 28 days after bleomycin administration. The inflammation markers that were measured in this study were the influx of leukocytes (neutrophils, macrophages and lymphocytes) into the airways, and the induction of the cytokines MCP-1 and MCP-3 (where MCP stands for Monocyte Chemoattractant Protein). Tables 2, 3, 4 and 5 show the total cell count, neutrophil count, macrophage count and the lymphocyte count, respectively, from the treated rats at 14 days and 28 days (Values for cell numbers are ×10⁶). The abbreviation SEM stands for Standard Error of the Mean. In Tables 2 thru 8 the t-test was an unpaired, two-tailed, t-test performed using a GraphPad Prism version 5.01 for Windows (GraphPad Software, San Diego, Calif. USA).

As shown in Tables 3, 4 and 5, the orally administered test compound inhibited the influx of neutrophils, macrophages and lymphocytes into the airways.

TABLE 2
Total Cell Count

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 1.835 | 4.020 | 1.988 | 1.482 |
| SEM | 0.375 | 0.629 | 0.334 | 0.188 |
| T-Test | vs. vehicle | p = 0.0104 | | |
| | vs. bleo | — | p = 0.0128 | p = 0.0017 |
| | control | | | |
| | 28 day | | | |
| Average | 1.335 | 2.320 | 2.276 | 2.207 |
| SEM | 0.325 | 1.143 | 0.630 | 0.445 |
| T-Test | vs. vehicle | p = 0.4214 | | |
| | vs. bleo | — | p = 0.9737 | p = 0.9265 |
| | control | | | |

TABLE 3
Neutrophil Count

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 0.003 | 0.020 | 0.006 | 0.005 |
| SEM | 0.001 | 0.007 | 0.002 | 0.002 |
| T-Test | vs. vehicle | p = 0.0256 | | |
| | vs. bleo | — | p = 0.0594 | p = 0.0468 |
| | control | | | |
| | 28 day | | | |
| Average | 0.002 | 0.022 | 0.012 | 0.008 |
| SEM | 0.001 | 0.016 | 0.006 | 0.002 |
| T-Test* | vs. vehicle | p = 0.2107 | | |
| | vs. bleo | — | p = 0.5392 | p = 0.3734 |
| | control | | | |

TABLE 4
Macrophage Count

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 0.367 | 0.812 | 0.415 | 0.285 |
| SEM | 0.071 | 0.120 | 0.087 | 0.037 |
| T-Test | vs. vehicle | p = 0.0065 | | |
| | vs. bleo | — | p = 0.0178 | p = 0.0009 |
| | control | | | |
| | 28 day | | | |
| Average | 0.278 | 0.455 | 0.442 | 0.476 |
| SEM | 0.061 | 0.222 | 0.118 | 0.106 |
| T-Test | vs. vehicle | p = 0.4549 | | |
| | vs. bleo | — | p = 0.9603 | p = 0.9302 |
| | control | | | |

TABLE 5
Lymphocyte Count

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 0.002 | 0.006 | 0.002 | 0.003 |
| SEM | 0.001 | 0.002 | 0.001 | 0.001 |
| T-Test | vs. vehicle | p = 0.0800 | | |
| | vs. bleo | — | p = 0.1076 | p = 0.2498 |
| | control | | | |
| | 28 day | | | |
| Average | 0.003 | 0.007 | 0.007 | 0.004 |
| SEM | 0.002 | 0.004 | 0.003 | 0.001 |
| T-Test | vs. vehicle | p = 0.3432 | | |
| | vs. bleo | — | p = 0.8886 | p = 0.5416 |
| | control | | | |

Additionally, MCP-1 and MCP-3 levels in the rat Bronchoalveolar Lavage Fluid (BAL) were measured using Luminex xMap Technology CS1000 (Austin, Tex.) and a Procarta Cytokine Assay Kit (rat 15-plex) manufactured by Panomics, Inc. (Fremont, Calif.) that included analytes for: IL-1α, IL-1β, IL-6, TNFα, KC, MCP-1, MCP-3, MIP-1α, RANTES, VEGF, TGFb, IL-4, IL-10, IFNg, and GM-CSF. Three replicate assay wells (n=3) were analyzed for each experimental sample following the manufacturer's instructions. Quantitation of each analyte was determined against standard curves using MiraiBio's (Alameda, Calif.) Masterplex QT v 3.0 software. For all samples, background signals in the absence of BAL were determined and subtracted from signals obtained in the presence of BAL to obtain the net signal. Statistical significance of biological studies was tested with a two-tailed Student t-test. Tables 6 and 7 show the concentrations of cytokines MCP-1 and MCP-3, respectively, in the airways of the treated rats, expressed in pg/mL. The test compound inhibited the production of MCP-1 and MCP-3 in the lungs of the treated rats.

TABLE 6
MCP-1

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 14.912 | 112.825 | 66.122 | 13.838 |
| SEM | 1.802 | 19.911 | 17.013 | 2.376 |
| T-Test | vs. vehicle | p < 0.0001 | | |
| | vs. bleo | — | p = 0.0811 | p < 0.0001 |
| | control | | | |
| | 28 day | | | |
| Average | 7.887 | 10.556 | 12.800 | 11.844 |
| SEM | 1.998 | 3.538 | 3.404 | 2.590 |
| T-Test | vs. vehicle | p = 0.5145 | | |
| | vs. bleo | — | p = 0.6498 | p = 0.7703 |
| | control | | | |

TABLE 7
MCP-3

| | | | Bleomycin + | Bleomycin + |
|---|---|---|---|---|
| | Vehicle | Bleomycin | 10 mg/Kg | 30 mg/Kg |
| | Control | Only | Test Compound | Test Compound |
| | 14 day | | | |
| Average | 4.484 | 87.706 | 37.987 | 5.668 |
| SEM | 1.321 | 18.500 | 11.445 | 1.015 |
| T-Test | vs. vehicle | p < 0.0001 | | |
| | vs. bleo | — | p = 0.0269 | p < 0.0001 |
| | control | | | |
| | 28 day | | | |
| Average | 2.409 | 9.777 | 7.075 | 6.497 |
| SEM | 0.550 | 2.919 | 2.704 | 1.556 |
| T-Test | vs. vehicle | p = 0.0168 | | |
| | vs. bleo | — | p = 0.5004 | p = 0.3266 |
| | control | | | |

Collagen deposition was used as a marker for the fibrotic response induced by bleomycin. The collagen assay was based on the assay described in Sykes B, et al., The estimation of two collagens from human dermis by interrupted gel electrophorsis, Biochem Biophys Res Commun. 72:1472-1480 (1976). Lung tissue from the treated rats was subjected to an overnight pepsinization, that was limited by the ambient temperature, by incubating 25 mg sample of powdered lung tissue with 0.25 ml of a 2.0 mg/ml solution of pepsin in 0.5N acetic acid at room temperature. 25 μL aliquots of the pepsin digests were reduced with dithiothreitol (DTT) and then subjected to SDS-polyacrylamide gel electrophoresis (PAGE) on 4-20% gradient gels. Gels were then stained with Coomassie Blue and stained bands migrating coincident with type I collagen standards were compared by densitometry to the Coomassie stained collagen standards in order to quantitate collagen content. Under the reducing conditions employed in the analysis, the quantitated lung sample gel bands represented the alpha 1 and 2 chains of type I collagen and the alpha 1 chain of type Ill collagen. Blots were scanned with a BioRad GS-800 Scanner and their density was quantitated using “Quantity One” computer software from BioRad. Results were averaged for each treatment group and expressed as micrograms of collagen/mg of lung tissue. Table 8 shows the amount of collagen deposition in the airways of the treated rats during the fibrotic phase of the response to bleomycin (Collagen concentration is expressed in units of pg/mg lung tissue). The orally administered test compound inhibited the deposition of collagen during the fibrotic phase of the bleomycin response.

TABLE 8
Collagen Response

| | 28 day | | | |
|---|---|---|---|---|
| | | | Bleomycin + | Bleomycin + |
| | Vehicle | Bleomycin | 10 mg/Kg | 30 mg/Kg |
| | Control | Only | Test Compound | Test Compound |
| Average | 3.379 | 5.766 | 2.353 | 3.891 |
| SEM | 0.336 | 0.815 | 0.359 | 0.598 |
| T-Test | vs. vehicle | p = 0.0233 | | |
| | vs. bleo | — | p = 0.0018 | p = 0.0847 |
| | control | | — | |

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method of treating or preventing interstitial lung diseases comprising administering to a human being (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof.

2. The method of claim 1, wherein the method is treating or preventing one or more of idiopathic pulmonary fibrosis, connective tissue or autoimmune disease-related pulmonary fibrosis, hypersensitivity pneumonitis, sarcoidosis, eosinophilic granuloma, Langerhan's cell histiocytosis, chronic eosinophilic pneumonia, Wegener's granulomatosis, idiopathic pulmonary hemosiderosis, bronchiolitis obliterans, scleroderma and lymphangioleiomyomatosis.

3. The method of claim 1, wherein the method is treating or preventing idiopathic pulmonary fibrosis, connective tissue or autoimmune disease-related pulmonary fibrosis, hypersensitivity pneumonitis, sarcoidosis, eosinophilic granuloma, Langerhan's cell histiocytosis, chronic eosinophilic pneumonia, Wegener's granulomatosis, idiopathic pulmonary hemosiderosis, bronchiolitis obliterans, scleroderma or lymphangioleiomyomatosis.

4. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by aerosol delivery.

5. The method of claim 1, wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered by oral delivery.

6. The method of claim 1, further comprising administration of an additional therapeutic agent to the human being.

7. A pharmaceutical composition for the treatment of interstitial lung disease comprising (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof and one or more pharmaceutically acceptable carrier.

8. The pharmaceutical composition of claim 7, for the treatment or prevention of idiopathic pulmonary fibrosis, connective tissue or autoimmune disease-related pulmonary fibrosis, hypersensitivity pneumonitis, sarcoidosis, eosinophilic granuloma, also known as Langerhan's cell histiocytosis, chronic eosinophilic pneumonia, Wegener's granulomatosis, idiopathic pulmonary hemosiderosis, bronchiolitis obliterans, scleroderma or lymphangioleiomyomatosis.

9. The pharmaceutical composition of claim 7, for the treatment or prevention of idiopathic pulmonary fibrosis.

10. The pharmaceutical composition of claim 7, wherein the composition is an aerosol formulation.

11. The pharmaceutical composition of claim 7, wherein the composition is an oral formulation.

12. The pharmaceutical composition of claim 7, further comprising an additional therapeutic agent.

13. The method of claim 1 wherein the (R)-N-((2S,3S)-2-(fluoromethyl)-2-hydroxy-5-oxo-tetrahydrofuran-3-yl)-5-isopropyl-3-(isoquinolin-1-yl)-4,5-dihydroisoxazole-5-carboxamide or a pharmaceutically acceptable salt thereof is administered to the human being in an amount of from 1 mg/kg to 500 mg/kg per day.
